(19) United States
(12) Patent Application Publication
(10) Pub. No.: US 2024/0140777 A1
(43) Pub. Date: May 2, 2024

(54) CLEANING ASSEMBLY FOR A BEVERAGE DISPENSER

(71) Applicant: MANITOWOC FOODSERVICE COMPANIES, LLC, NEW PORT RICHEY, FL (US)

(72) Inventor: Kenneth L. LUNDBERG, Land O Lakes, FL (US)

(73) Assignee: MANITOWOC FOODSERVICE COMPANIES, LLC, NEW PORT RICHEY, FL (US)

(21) Appl. No.: 17/978,751

(22) Filed: Nov. 1, 2022

(51) Int. Cl. B67D 1/07; A61L 2/18; A61L 2/26; B08B 3/08

(52) U.S. Cl. CPC B67D 1/07; A61L 2/18; A61L 2/26; B08B 3/08; A61L 2202/15; A61L 2202/17; B67D 2001/075

(57) ABSTRACT

A cleaning system for a drink dispensing device comprising a cleaner canister coupled to a water source and a cleaner CFValve (solenoid valve) coupled to the water source which provides a first water flow to the cleaner canister, the cleaner canister configured to provide a cleaner solution to one or more parts of the drink dispensing device. The cleaning system is further comprising a sanitizer canister coupled to the water source and a sanitizer CFValve (solenoid valve) coupled to the water source which provides a second water flow to the sanitizer canister, the sanitizer canister configured to provide a sanitizer solution to one or more parts of the drink dispensing device. The cleaning system is further comprising a water flush device coupled to the water source and a water flush CFValve (solenoid valve) coupled to the water source which provides a third water flow to the one or more parts of the drink dispensing device.

### BACKGROUND OF THE DISCLOSURE

### 1. Field of the Disclosure

The present disclosure is directed to beverage dispensers. More particularly, the present disclosure relates to a cleaning system and assembly for a beverage dispenser.

### 2. Description of the Related Art

Beverage devices are available that mix and/or blend ice and flavored ingredients together to form a frozen beverage, for example, smoothies, frappes, Frappuccino® drinks, daquiris, and other beverages. These devices require periodic cleaning to ensure flavor consistency and to maintain safety. Such cleaning undesirably requires operators of the beverage devices to remember to perform the cleaning as well as connect the beverage devices to the cleaning liquid and/or sanitizing liquid sources each time the beverage devices are cleaned.

In an existing bucket and pump system, measured chemicals are poured into a bucket and filled with water to the correct level mark on the bucket and stirred to mix. This bucket is then carried to the front of the machine and pumped through to clean and sanitize. There are many chances for error in measurement of chemical and/or water in this procedure. Further skin contact of chemicals and, carry bucket spills etc. are problematic. In addition to a ratio control problem, there is a time issue and quality control issues. For example, time between cleaning, as well as time for solution to be present in plumbing to be effective. Furthermore, employee may just skip doing it as it is difficult to do, especially in a crowded store. Another issue is inconsistencies on when and how the device is cleaned. For example, a first employee may clean the system at the appropriate time but not at the appropriate ratios. A second employee may not clean the system at all. However, a third employee may clean the system at the appropriate times with the appropriate ratio. In another system, a ChemStation type—filling a bucket with chemicals and water, each by weight, has the same issues noted above. In another example, an Ecolab type process may be used by filling a bucket by a timed run of a peristaltic pump, and has the same issues noted above. In addition, weekly service to “top off” large storage tank containing a mixed solution, gravity feed to bucket, has the same issues noted above.

Thus, existing systems for cleaning beverage, ice cream, ovens and other food equipment are very reliant on labor and efficacy of the cleaning, and can be impacted by the employee's calculation of dilution factor, and remembering/choosing to run the daily, hourly and weekly cleaning cycles. This is subject to serious error. With increased focus on cleaning for quality and safety and with increased awareness by recent global events, restaurants, convenient stores, fast food, institutional purveyors, and regulators need better guarantees that equipment is being cleaned appropriately and to the standards required.

Additionally, there is an opportunity for much improved environmental impact and business system impact by providing high and ultra-high ratio concentrates that are diluted on demand reducing package size, shipping size and frequency and dramatically reducing the carbon footprint and costs of delivering cleaning, sanitizing, descaling and other concentrates.

Accordingly, it has been determined by the present disclosure that there is a continuing need for a device that overcomes, alleviates, and/or mitigates one or more of the aforementioned issues, and other deleterious effects of prior devices.

### SUMMARY OF THE DISCLOSURE

Thus, there is an opportunity for much improved environmental impact and business system impact by providing high and ultra-high ratio concentrates that are diluted on demand, reducing package size, shipping size and frequency, and dramatically reducing the carbon footprint and costs of delivering cleaning, sanitizing, descaling and other concentrates.

In general, an embodiment of the disclosure is directed to a cleaning assembly, which is provided for a beverage dispenser. Further, this disclosure illustrates a cleaning (e.g., cleaning, sanitizing, and/or rinsing) system for a beverage dispensing device.

The user can use the cleaning system for a drink dispensing device comprising a cleaner canister coupled to a water source; and a cleaner CFValve (solenoid valve) coupled to the water source which provides a first water flow to the cleaner canister, the cleaner canister configured to provide a cleaner solution to one or more parts of the drink dispensing device. The cleaning system further comprises a sanitizer canister coupled to the water source and a sanitizer CFValve (solenoid valve) coupled to the water source which provides a second water flow to the sanitizer canister, the sanitizer canister configured to provide a sanitizer solution to one or more parts of the drink dispensing device.

None of the systems now in service have the clean in place (CIP) feature, i.e., an ultra-high ratio (UHR) concentrate internal to the machine using the same city water supply that does not require an additional water connection to the machine, and wherein the machines' own computer will turn the cleaning system on and off as required. These may all be in one or more embodiments disclosed herein.

The clean in place method of sanitation is internal to the machine being cleaned. One or more UHR canisters of concentrated chemical cleaning liquid may be calibrated to perform the cleaning cycle for a period of weeks or months without having to be replaced. The canisters are mounted internal to the machine, plumbed into the machine's water supply source, to be cycled on and off as necessary by the machine's computer system. When the sealed, single use, canister is empty the machine may stop serving until the empty canister[s] is disconnected at the dry break and replaced with a new cartridge.

The same clean in place system can be situated beside, behind, beneath the machine or even in the backroom of the facility. It can be automatically run by its own controller/computer/timer in the same way the internal-inside machine is run.

One example is a double bag design. The canister may be constructed with a double bag inside a rigid body if there is a chemical compatibility. The interior bag, which is contained in the outer bag, contains the cleaning chemicals. The water to supply the pressure and to mix with the solution is fed in between the two bags and expands outward to be contained by the outer rigid body. There is a vent in the rigid body to allow the air to escape as the bag is filled. The benefit of the double bag system is that there is a vacuum between the two bags and there is no chance of air being compressed, changing the ratio of the concentrate to water mixture.

Another example is a single bag design. The single bag design uses a bag inside a rigid canister. The water supply fills the canister and compresses the bag.

The canister may be assembled with all components built into the cap—check valves, mixing chamber, orifice, etc. This makes for simple assembly and reduced costs (this is different than current UHR designs). Additionally, there may be dry break to the canister that allow for a dripless connection on the inlet water and the diluted outbound solution. This is an advantage for when the canister is replaced when it is empty. The inlet and outlet Dole feature is designed so that it can be placed at any angle to accommodate where it resides in the machine. Dole fittings allow a universal installation as the supply/discharge would be in any direction.

A system can have single or multiple canisters to allow for different chemicals (i.e.: cleaner, sanitizer, de-scaler) and can also have multiple different cycles utilizing one or more of the chemicals for varying different lengths of time as needed to clean for different products (i.e.: coffee, milk, juice, ice cream, steam oven, etc.) Multiple canisters would be an advantage because the cleaning for different products (for example, juice or milk) may require a different chemical or a different time to flush.

All of the cycles are controlled either through the controller of the existing equipment or by an external controller/computer built into the CFV(SV)-UHR-CIP kit. For example, a smoothie machine may run 4-hour sanitation, daily clean (short) and weekly clean (long) cycles. The built-in system will shut down the machine every four hours and run the four-hour cycle, and once a day to run the daily cycle and then once a week for the weekly cycle. The machine will be inoperable during the cleaning cycle and will automatically be flushed with clean water after the cycle. Furthermore, there is a total dissolved solids (conductivity) meter and/or sensors that measures the total dissolved solids (conductivity) in the city water and then the dissolved solids in the mixed chemical. This will tell the system (whether internal or external) whether the chemical amount is on target and will provide a sold-out feature to notify of the need for chemical replacement. Also, the “sold out” feature can be utilized to shut down the entire machine so there is no way for an employee to allow a “dirty” machine to dispense/cook/mix product.

The canisters can be designed to be single use or recyclable. However, they are designed to be plug and play to the system (again through the dry break/dripless connections). The dry break connections are “pokey-oked” so that the operator/employee can NOT connect the inlet to the outlet in error and cannot connect the wrong chemical cartridge to the wrong inlet/outlet port (i.e., cleaner to the sanitizer port).

The design of the orifice tube connection to the bag and to the manifold provides a streamline approach that doesn't allow kinking or compression in the canister.

Because the inlet water is controlled by an electronically actuated CFValve (solenoid valve), it provides an essentially constant pressure and the ability to electronically actuate based on time of day and length of desired clean. Furthermore, the addition of the CFiVe for the flush and TDS (conductivity) circuit allows the system to be automatically flushed after use and force the TDS (conductivity) sensor to take a baseline water dissolved solids reading to apply the Delta calculation to ensure the proper dilution/strength of the chemical and sold-out feature.

In light of the foregoing, it will now be appreciated by those skilled in the art that the present disclosure embodies a number of significant advantages, the foremost being the automatic pressure responsive control of fluid flow between a variable pressure source and an applicator from which the fluid is to be applied in a substantially uniform manner. The regulating valve is designed for low-cost mass production, having a minimum number of component parts, the majority of which can be precision molded and automatically assembled.

In one embodiment, a cleaning system for a drink dispensing device includes: a cleaner canister coupled to a water source; a cleaner CFValve (solenoid valve) coupled to the water source which provides a first water flow to the cleaner canister. The cleaner canister may provide a cleaner solution to one or more parts of the drink dispensing device.

In another example, the cleaning system may include a sanitizer canister coupled to the water source and a sanitizer CFValve (solenoid valve) coupled to the water source which provides a second water flow to the sanitizer canister. The sanitizer canister may provide a sanitizer solution to one or more parts of the drink dispensing device. In another example, the cleaning system may include a water flush device coupled to the water source and a water flush CFValve (solenoid valve) coupled to the water source which provides a third water flow to the one or more parts of the drink dispensing device.

In another example, the cleaning system may include an inlet dry breaking (dripless) fitting and an outlet dry breaking fitting on the sanitizer canister. In another example, the cleaning system may include an inlet dry breaking fitting and an outlet dry breaking fitting on the cleaner canister. In another example, the cleaning system may include a total dissolved solids (conductivity) device which measures an inlet total dissolved solids (conductivity) and an outlet total dissolved solids (conductivity). In another example, the cleaning system may include a sanitizer canister coupled to the water source and a sanitizer CFValve (solenoid valve) coupled to the water source which provides a second water flow to the sanitizer canister. The sanitizer canister may provide a sanitizer solution to one or more parts of the drink dispensing device. A water flush device coupled to the water source and a water flush CFValve (solenoid valve) coupled to the water source which provides a third water flow to the one or more parts of the drink dispensing device. A total dissolved solids (conductivity) device which measures an inlet total dissolved solids (conductivity) and an outlet total dissolved solids (conductivity). In another example, the cleaning system may include a sanitizer canister coupled to the water source and a sanitizer CFValve (solenoid valve) coupled to the water source which provides a second water flow to the sanitizer canister. The sanitizer canister may provide a sanitizer solution to one or more parts of the drink dispensing device; a water flush device coupled to the water source and a water flush CFValve (solenoid valve) coupled to the water source which provides a third water flow to the one or more parts of the drink dispensing device. A total dissolved solids (conductivity) device which measures an inlet total dissolved solids (conductivity) and an outlet total dissolved solids (conductivity). An inlet dry breaking fitting and an outlet dry breaking fitting on the sanitizer canister. An inlet dry breaking fitting and an outlet dry breaking fitting on the cleaner canister. A controller that controls one or more ratios based on the inlet total dissolved solids (conductivity) and the outlet total dissolved solids (conductivity). In another example, one or more of the cleaner CFValve (solenoid valve), the sanitizer CFValve (solenoid valve), and the water flush CF Valve may maintain a relative constant flow of fluid from a variable pressure fluid supply to a fluid outlet, the CF Valve including: a) a valve housing having an inlet port and an outlet port adapted to be connected to the variable pressure fluid supply and the fluid outlet; b) a diaphragm chamber interposed between the inlet port and the outlet port; c) a cup contained within the diaphragm chamber; d) a diaphragm closing the cup; e) a piston assembly secured to a center of the diaphragm, the piston assembly having a cap and a base; f) a stem projecting from the cap through a first passageway in a barrier wall to terminate in a valve head; and g) a spring in the cup co-acting with the base of the piston assembly for urging the diaphragm into a closed position, and the spring being responsive to fluid pressure above a predetermined level to adjust a size of a control orifice. In another example, one or more of the cleaner CFValve (solenoid valve), the sanitizer CFValve (solenoid valve), and the water flush CFValve (solenoid valve) is configured to maintain a relative constant flow of fluid from a variable pressure fluid supply to a fluid outlet, the CF Valve including: a base having a wall segment terminating in an upper rim, and a projecting first flange; a cap having a projecting ledge and a projecting second flange, the wall segment of the base being located inside the cap with a space between the upper rim of the base and the projecting ledge of the cap; a barrier wall subdividing an interior of a housing into a head section and a base section; a modulating assembly subdividing the base section into a fluid chamber and a spring chamber; an inlet in the cap for connecting the head section to a fluid source; a port in the barrier wall connecting the head section to the fluid chamber, the port being aligned with a central first axis of the CF Valve; an outlet in the cap communicating with the fluid chamber, the outlet being aligned on a second axis transverse to the first axis; a stem projecting from the modulating assembly along the first axis through the port into the head section; a diaphragm supporting the modulating assembly within the housing for movement in opposite directions along the first axis, a spring in the spring chamber, the spring being arranged to urge the modulating assembly into a closed position at which the diaphragm is in sealing contact with the barrier wall, and the spring being responsive to fluid pressure above a predetermined level to adjust a size of a control orifice.

In another embodiment, a cap for a canister may include: a CFValve (solenoid valve) coupled to a cleaning solution source; a tube coupled to the CFValve (solenoid valve) to transport a cleaning solution; and a tube outlet area to deliver the cleaning solution.

In another example, the tube has a first length, and the delivered cleaning solution has a first cleaning solution concentration based on the first length. In another example, the tube has a second length, and the delivered cleaning solution has a second cleaning solution concentration based on the second length. In another example, a second tube that has a second length and the delivered cleaning solution has a second cleaning solution concentration based on the second length and wherein the tube has a first length, and the delivered cleaning solution has a first cleaning solution concentration based on the first length and wherein the first cleaning solution concentration is different than the second cleaning solution concentration.

In another embodiment, a canister may include: a body with an inlet and an outlet; a cap including a mixing chamber, one or more orifices, and one or more check valves; the inlet coupled to the cap, a CFValve (solenoid valve), and a first total dissolved solids (conductivity) sensor; and the outlet coupled to the cap and a second total dissolved solids (conductivity) sensor, the outlet may deliver a flow from the canister.

In another example, the flow from the canister is modified based on data delivered to a controller from at least one of the first total dissolved solids (conductivity) sensor and the second total dissolved solids (conductivity) sensor. In another example, the canister may include a tube with a first length from the CFValve (solenoid valve) to the outlet where a concentrate of the flow is determined by the first length. In another example, the CFValve (solenoid valve) may maintain a relative constant flow of fluid from a variable pressure fluid supply to a fluid outlet, the CF Valve including: a) a valve housing having an inlet port and an outlet port adapted to be connected to the variable pressure fluid supply and the fluid outlet; b) a diaphragm chamber interposed between the inlet port and the outlet port; c) a cup contained within the diaphragm chamber; d) a diaphragm closing the cup; e) a piston assembly secured to a center of the diaphragm, the piston assembly having a cap and a base; f) a stem projecting from the cap through a first passageway in a barrier wall to terminate in a valve head; and g) a spring in the cup contacting with the base of the piston assembly for urging the diaphragm into a closed position, and the spring being responsive to fluid pressure above a predetermined level to adjust a size of a control orifice. In another example, the CFValve (solenoid valve) is configured to maintain a relative constant flow of fluid from a variable pressure fluid supply to a fluid outlet, the CF Valve including: a base having a wall segment terminating in an upper rim, and a projecting first flange; a cap having a projecting ledge and a projecting second flange, the wall segment of the base being located inside the cap with a space between the upper rim of the base and the projecting ledge of the cap; a barrier wall subdividing an interior of a housing into a head section and a base section; a modulating assembly subdividing the base section into a fluid chamber and a spring chamber; an inlet in the cap for connecting the head section to a fluid source; a port in the barrier wall connecting the head section to the fluid chamber, the port being aligned with a central first axis of the CF Valve; an outlet in the cap communicating with the fluid chamber, the outlet being aligned on a second axis transverse to the first axis; a stem projecting from the modulating assembly along the first axis through the port into the head section; a diaphragm supporting the modulating assembly within the housing for movement in opposite directions along the first axis, a spring in the spring chamber, the spring being arranged to urge the modulating assembly into a closed position at which the diaphragm is in sealing contact with the barrier wall, and the spring being responsive to fluid pressure above a predetermined level to adjust a size of a control orifice.

In one embodiment, a liquid dispensing system may include: a container enclosing a chamber; a flexible bag in the chamber; a first liquid contained in the bag; a first and second conduits in the chamber, the first conduit connecting the chamber to an outlet port in the container, the second conduit connecting the bag to the first conduit; and a supply source for introducing a pressurized second liquid into the chamber, the first conduit serving to direct an existing flow of the second liquid from the chamber to the outlet port, with the pressurized second liquid serving to collapse the bag and expel the first liquid contained therein via the second conduit to the first conduit for mixture with the exiting flow of the second liquid.

In another example, the supply source includes a constant flow valve located externally of the container. In another example, the liquid dispensing system includes a check valve in the first conduit for preventing a reverse flow of liquid into the chamber. In another example, the liquid dispensing system includes a check valve on the second conduit for preventing a reverse flow of liquid into the bag. In another example, the liquid dispensing system includes the first conduit includes a metering orifice. In another example, the liquid dispensing system has a second conduit that is a flexible tube. In another example, the check valves comprise duckbill valves. In another example, the supply source is connected to the container by a dry break quick connect coupling. In another example, the first conduit communicates with an upper region of the chamber and the pressurized liquid is introduced into a lower region of the chamber via an inlet port in the container. In another example, the liquid dispenses includes a third open ended bypass conduit arranged between the interior of the container and the bag, the bypass conduit extending from the lower region to the upper region of the chamber.

In another embodiment, a liquid dispensing system may include: a container enclosing a chamber having upper and lower regions; a flexible bag in the chamber, the bag extending vertically between the upper and lower regions; a first liquid contained in the bag; a first, a second and a third conduits in the chamber, the first conduit leading to an outlet port in the container, the second conduit connecting the bag to the first conduit; and a supply source for introducing a pressurized second liquid into the chamber and separately into the third conduit for delivery to the first conduit, the first conduit serving to direct an existing flow of the second liquid to the outlet port, with the pressurized second liquid in the chamber serving to collapse the bag and expel the first liquid contained therein via the second conduit to the first conduit for mixture with the exiting flow of the second liquid. The drinking liquid may be replaced with cleaning and/or sanitizer or anything else in this disclosure and still utilize any of the above-referenced elements and/or configuration.

In another example, the supply source includes a constant flow valve located externally of the container. In another example, the liquid dispensing system includes a check valve in the first conduit for preventing a reverse flow of liquid into the chamber. In another example, the liquid dispensing system includes a check valve on the second conduit for preventing a reverse flow of liquid into the bag. In another example, the first conduit includes a metering orifice. In another example, the pressurized liquid is introduced into a T-fitting in the chamber, the T-fitting having one branch communicating with the third conduit and having another branch communicating with the chamber.

In one embodiment, a liquid dispensing system may include: a first container enclosing a chamber; a flexible container in the chamber; a first liquid contained in the flexible container; a first conduit and a second conduit in the chamber, the first conduit connecting the chamber to an outlet port in the first container, the second conduit connecting the flexible container to the first conduit where the second conduit is coupled to the flexible container at a flexible container outlet location, wherein the second conduit is connected to the chamber via an orifice and the output port; a supply source for introducing a pressurized second liquid into the chamber, the first conduit serving to direct an existing flow of the pressurized second liquid from the chamber to the outlet port, with the pressurized second liquid serving to collapse the flexible container and expel the first liquid contained therein via the second conduit to the first conduit for mixture with the exiting flow of the pressurized second liquid; and/or a third conduit arranged between an interior of the first container and the flexible container, the third conduit extending from a lower region to an upper region of the chamber and coupled to a T-fitting in the chamber.

In another example, the supply source includes a constant flow valve located external of the first container. In another example, the liquid dispensing system may include a first check valve configured to prevent a reverse flow of liquid into the chamber or further comprising a second check valve on the second conduit for preventing a reverse flow of liquid into the flexible container. In another example, the first check valve or the second check valve may be duckbill valves. In another example, the first conduit includes the orifice. In another example, the second conduit comprises a flexible tube. In another example, the supply source is connected to the container by a dry break quick connect coupling. In another example, the first conduit communicates with the upper region of the chamber, and wherein the pressurized second liquid is introduced into the lower region of the chamber via an inlet port in the first container wherein the flexible container outlet location is located at a bottom part of the flexible container. In another example, the first conduit comprises the orifice fitted to an opposite end of an elbow fitting. In another example, the second conduit comprises a first check valve to provide a connection between the chamber and the outlet port.

In another embodiment, a liquid dispensing system may include: a first container enclosing a chamber; a flexible container in the chamber; a first liquid contained in the flexible container; a first conduit and a second conduit in the chamber, the first conduit connecting the chamber to an outlet port in the first container, the second conduit connecting the flexible container to the first conduit where the second conduit is coupled to the flexible container at a flexible container outlet location; a supply source for introducing a pressurized second liquid into the chamber, the first conduit serving to direct an existing flow of the pressurized second liquid from the chamber to the outlet port, with the pressurized second liquid serving to collapse the flexible container and expel the first liquid contained therein via the second conduit to the first conduit for mixture with the exiting flow of the pressurized second liquid; one or more check valves for preventing a reverse flow of liquid into the chamber or into the flexible container; and/or a third conduit arranged between an interior of the first container and the flexible container, the third conduit extending from a lower region to an upper region of the chamber and coupled to a T-fitting in the chamber.

In another example, the supply source includes a constant flow valve located external of the first container. In another example, the first conduit includes a metering orifice. In another example, the second conduit comprises a flexible tube. In another example, the supply source is connected to the first container by a dry break quick connect coupling. In another example, the first conduit communicates with the upper region of the chamber, and wherein the pressurized second liquid is introduced into the lower region of the chamber via an inlet port in the first container wherein the flexible container outlet location is located at a bottom part of the flexible container. In another example, the supply source is connected to the first container by a dry quick break connect coupling to a nipple structure. In another example, the liquid dispensing system may include a T-shaped fitting coupled to the third conduit and the lower region of the chamber.

In the above embodiments and examples, the drinking liquid may be replaced with cleaning and/or sanitizer or anything else described in this disclosure and still utilize any of the above-referenced elements and/or configuration.

In another example, the canister may be coupled to a drink dispensing system for one or more cleaning procedures.

The above and other objects, features, and advantages of the present disclosure will be apparent and understood by those skilled in the art from the following detailed description, drawings, and accompanying claims. As shown throughout the drawings, like reference numerals designate like or corresponding parts.

### BRIEF DESCRIPTION OF THE DRAWINGS

FIG. 1 is an illustration of a sanitation system, according to one embodiment.

FIG. 2 is another illustration of a sanitation system, according to one embodiment.

FIG. 3 is another illustration of a sanitation system, according to one embodiment.

FIG. 4 is an illustration of a bag-in-bottle sanitation system, according to one embodiment.

FIG. 5A is another illustration of a bag-in-bottle sanitation system, according to another embodiment.

FIG. 5B is another illustration of a bag-in-bottle sanitation system, according to another embodiment.

FIG. 6 is an illustration of a sanitation system, according to one embodiment.

FIG. 7A is an illustration of a molded device, according to one embodiment.

FIG. 7B is an illustration of a stand-alone mix manifold, according to one embodiment.

FIG. 8 is an illustration of a cap of a canister, according to one embodiment.

FIG. 9 is another illustration of a cap of a canister, according to one embodiment.

FIG. 10 is an illustration utilizing a CFValve (solenoid valve), according to one embodiment.

FIG. 11 is an illustration of a sanitation system, according to one embodiment.

FIG. 12 is another illustration of a sanitation system, according to one embodiment.

FIG. 13 is another illustration of a sanitation system, according to one embodiment.

FIG. 14 is another illustration of a sanitation system, according to one embodiment.

FIG. 15 is an illustration of a canister, according to one embodiment.

A component or a feature that is common to more than one drawing is indicated with the same reference number in each of the drawings.

### DESCRIPTION OF THE EMBODIMENTS

As herein employed, the term “constant flow valve” (solenoid valve) means a flow control valve of the type described, for example, in any one of U.S. Pat. Nos. 7,617,839; 6,026,850 or 6,209,578, the descriptions of which are herein incorporated by reference in their entirety. These types of valves are normally closed, are opened in response to pressures exceeding a lower threshold level, are operative at pressures between the lower threshold level and an upper threshold level to deliver liquids at a substantially constant pressures and are again closed at pressures above the upper threshold level.

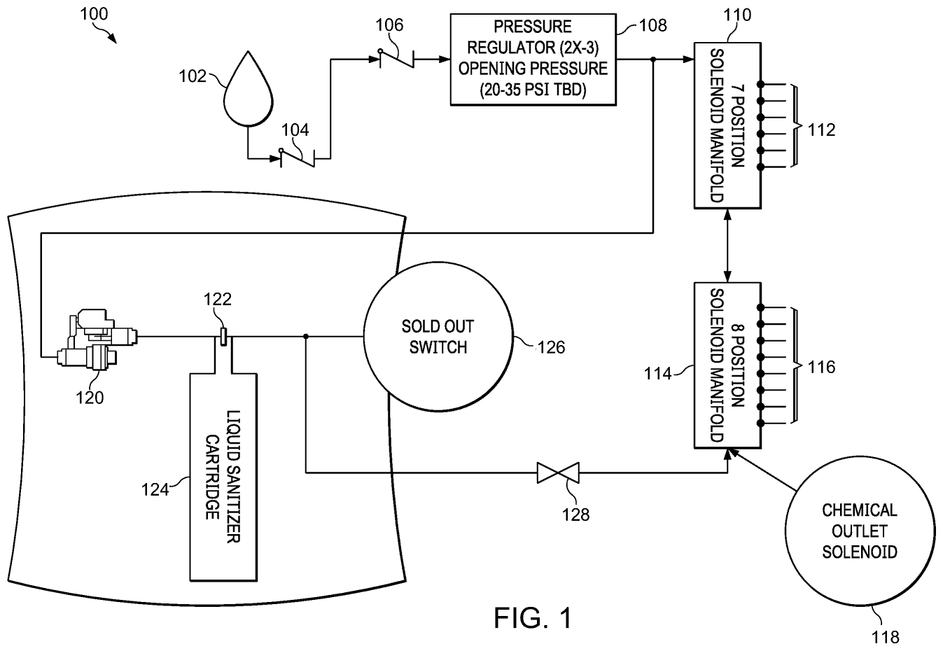

In FIG. 1, an illustration of a sanitation system is shown, according to one embodiment. A sanitation system 100 may include a water source 102, a flow meter 104, a pressure transducer 106, a pressure regulator 108, a first solenoid 110 with 7 positions 112 (any number of positions could be utilized), a second solenoid 114 with 8 positions 116 (any number of positions could be utilized), a chemical outlet solenoid 118, a CFValve (solenoid valve) 120, a water orifice 122 (and/or any other orifice), a sanitizer cartridge 124 (and/or container and/or canister), a sold out switch 126, and/or a check valve 128. In various examples, the pressure regulator is a 2X-3 regulator. In various examples, the opening pressure is between 20-35 PSI. In one example, the ratio is 375 to 1 and 0.5 min, 2-gallon target. In another example, the ratio is 525 to 1 and 0.5 minute, 2-gallon target. In various other examples, the ratio could be anywhere between 375-525 to 1 and a target of 1 gallon to 3 gallons per 0.5 minutes. In another example, the sold-out switch 126 is initiated based on the sanitizer cartridge being empty. For example, a ratio may be water to concentrate—1 part of concentrate to 300 parts of water.

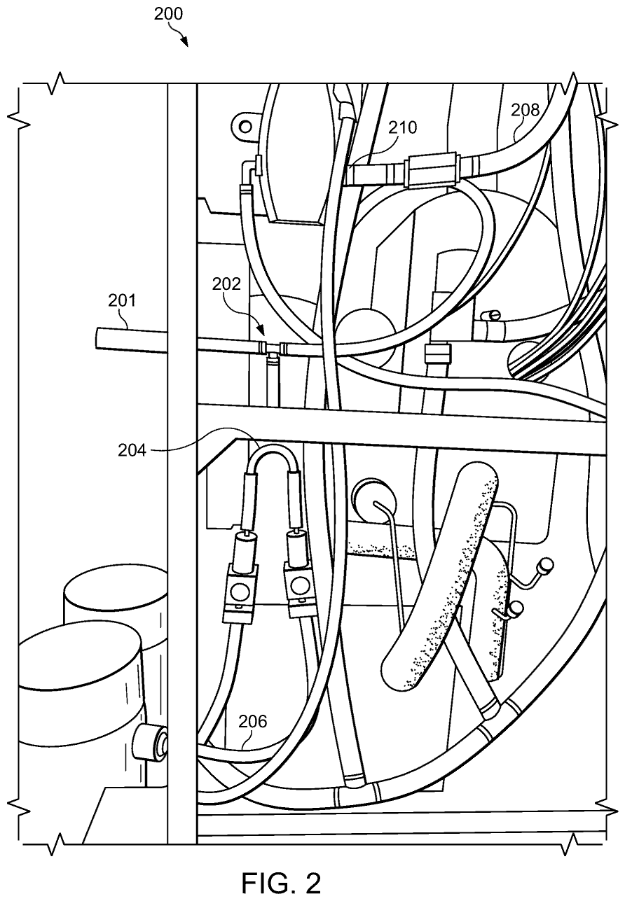

In FIG. 2, another illustration of a sanitation system is shown, according to one embodiment. A sanitation system 200 may include a water inlet 201, a water inlet tee 202, a Y water connection to CFives 204 (and/or one to sanitizer and/or one to cleaner), a mixed diluted sanitizer and cleaner outlet 206, a total dissolved solids (conductivity) meter 208 (and/or a flow meter and/or a temperature sensor), a mixed diluted sanitizer and cleaner outlet 210. In one example, there could be two lines where 1 would be dedicated for the sanitizer and 1 would be dedicated for the cleaner. In another example, the mixed diluted sanitizer and cleaner outlet 210 would connect to two check valves going to the Mac Valve Block. Further, to integrate a water wash down circuit and a manual pump circuit a manifold connection leading to the Mac Valve Block could be utilized. In one example of field retrofit, a hole would be needed to be cut in the rear panel to allow for out/in tubing. This could also be accomplished by replacing the rear panel. Further, a T and check valves may be placed on water inlet to Y fitting to CFives (e.g., CFValves (solenoid valves)). In addition, 2 tubes out of CFives with dry break to back of UHR cartridge may be utilized. Further, 2 tubes out of cartridge (dry break) into machine could be used which could be connected via check valves into Y fitting into cleaning line. Further, the locations of the CFives can be moved if needed.

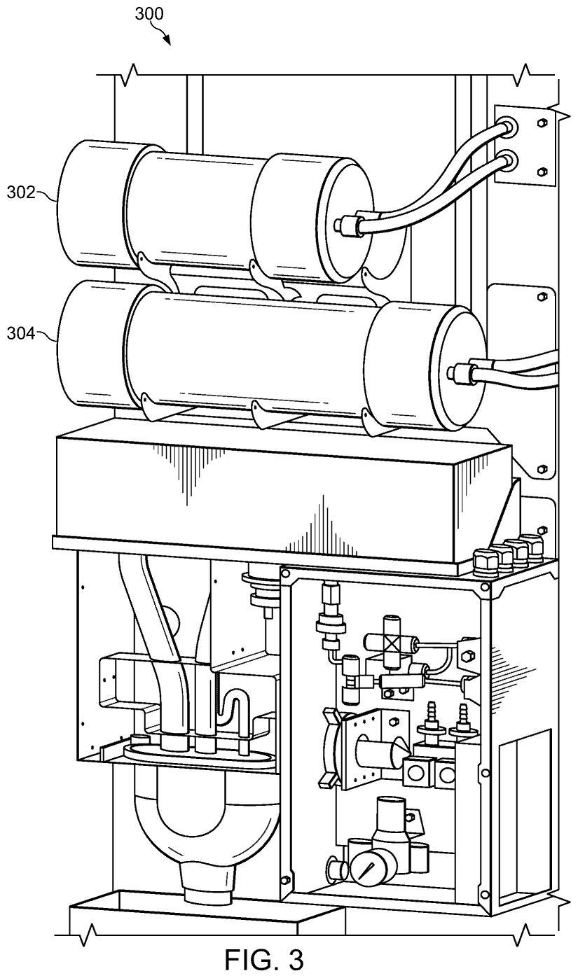

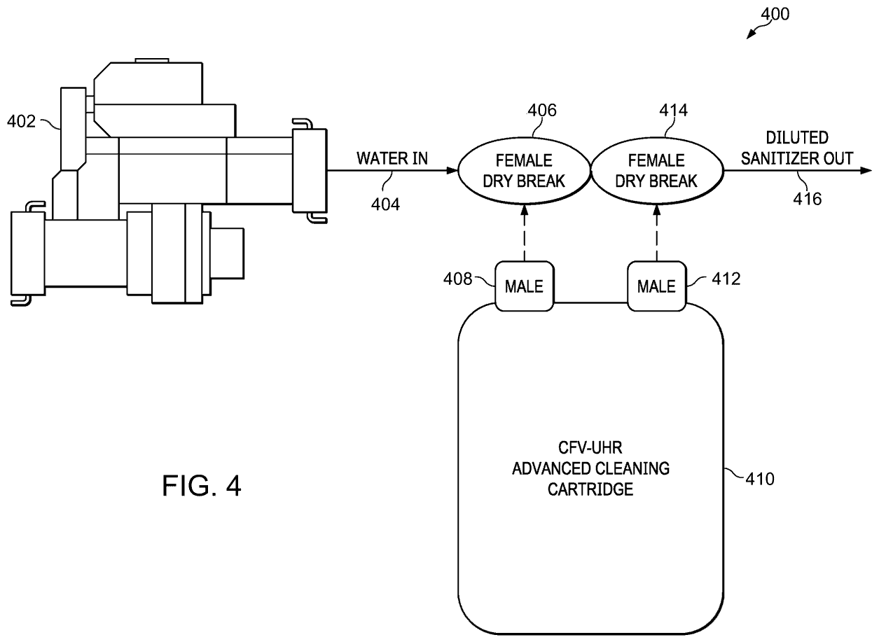

In FIG. 3, another illustration of a sanitation system is shown, according to one embodiment. A sanitation system 300 may include a first canister 302 (e.g., a sanitizer canister), a second canister 304 (e.g., a cleaning canister), and an Nth canister (e.g., anything in this disclosure) (not shown). The first canister 302, the second canister 304, and/or the Nth canister may be coupled to the drink dispensing system to allow water, sanitizer, and cleaning solutions to clean, sanitizes, and rinse any part of the drink dispensing system (see FIGS. 4-15).

In FIG. 4, an illustration of a bag-in-bottle sanitation system is shown, according to one embodiment. A sanitization system 400 may include a CFValve (solenoid valve) 402 (e.g., CFives) which provides a water in flow 404 to a first female dry break 406 which is coupled to a first male counterpart 408 on the cartridge 410 (e.g., cleaning, sanitizer, etc.) which is coupled to a second male counterpart 412 which is coupled to a second female dry break 414 which produces a diluted solution 416 (e.g., cleaning, sanitizer, etc.). This cartridge system can be utilized with any other system disclosed in this document.

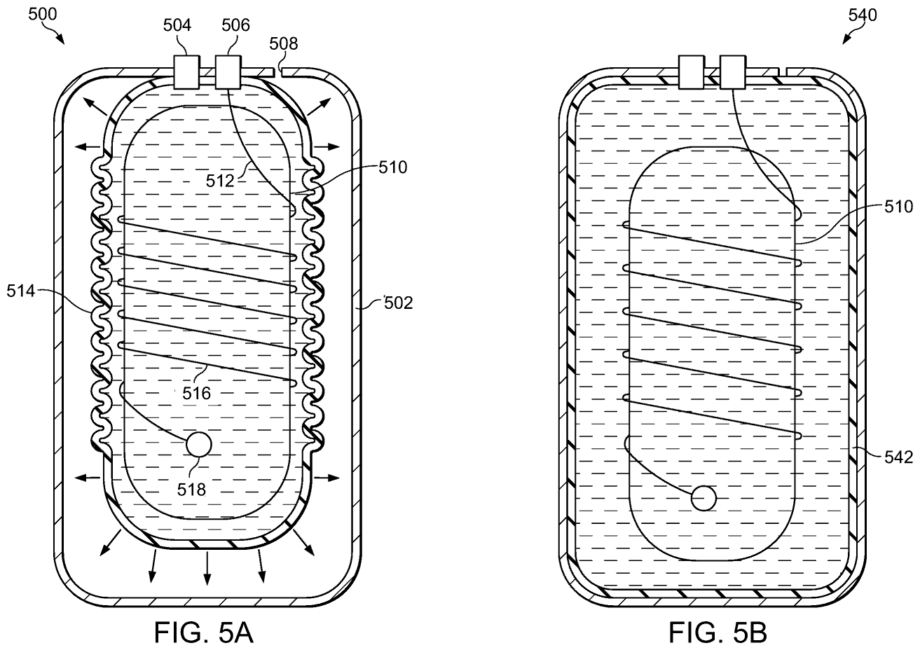

In FIG. 5A, another illustration of a bag-in-bottle sanitation system is shown, according to another embodiment. A sanitation system 500 may include a bag-in-bottle device 502 which may include an inlet 504, an outlet 506, a vent 508, a bag 510, a tube orifice 512, and/or an anchor 518. In one example, the outer bag is in a collapsed state 514 and the tube orifice is in a collapsed bag position 516. In FIG. 5B, another illustration of a bag-in-bottle sanitation system is shown, according to another embodiment. A sanitation system 540 may include a bag-in-bottle device 540 in an outer bag expanded state to file container 542.

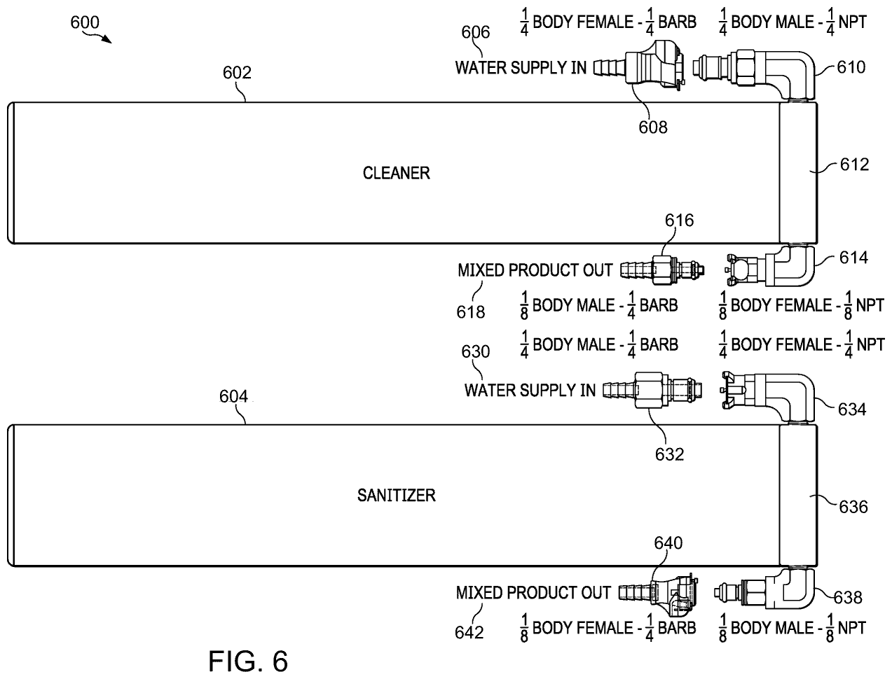

In FIG. 6, an illustration of a sanitation system is shown, according to one embodiment. A sanitation system 600 may include a cleaner canister 602, a sanitizer canister 604, a first water supply 606, a first inlet female break 608, a first inlet male break 610, a first manifold 612, a first outlet female break 614, a first outlet male break 616, a first mixed product out 618, a second water supply 630, a second inlet male break 632, a second inlet female break 634, a second manifold 636, a second outlet male break 638, a second outlet female break 640, and a second mixed product out 642. It should be noted that any cleaning product can be utilized in cleaner canister 602 and/or that any sanitizer product can be utilized in sanitizer canister 604. Further, based on the configuration of the first inlet female break 608, the first inlet male break 610, the first outlet female break 614, the first outlet male break 616, the second inlet male break 632, the second inlet female break 634, the second outlet male break 638, and the second outlet female break 640, the cleaner canister 602 and the sanitizer canister 604 cannot be placed in the wrong position. In other words, the sanitizer canister 604 cannot be located and/or coupled to the cleaner canister's 602 position because the female and male breaks are not aligned to do so. Further, the cleaner canister 602 cannot be located and/or coupled to the sanitizer canister's 604 location because the female and male breaks are not aligned to do so. The units are designed to not allow the cleaner to attach to the sanitizer (or active chemical A or B) and in both cases the water inlet to the outlet side and visa-versa cannot be connected.

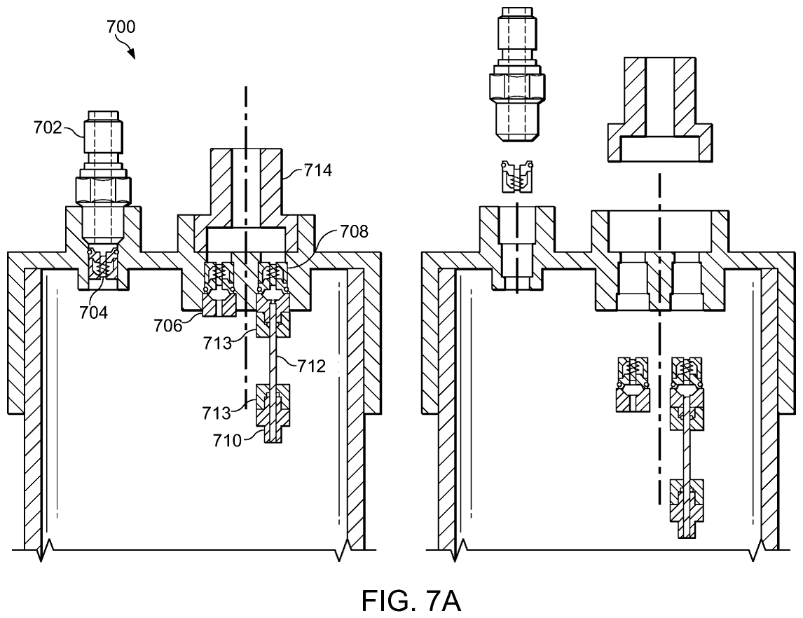

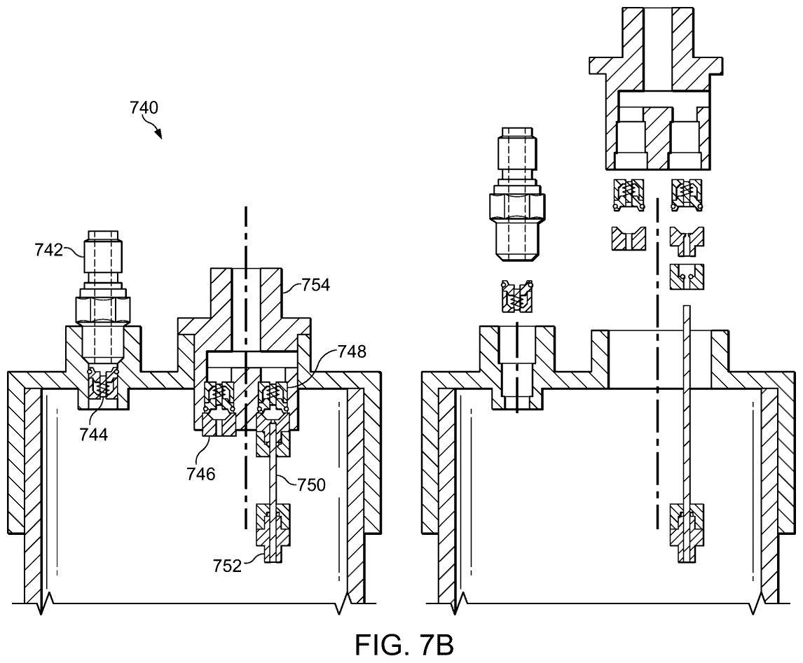

In FIG. 7A, an illustration of a molded device is shown, according to one embodiment. A device 700 may include an inlet quick connect dry break fitting 702, a water inlet check valve 704, a water outlet check orifice 706, a check valve retainer 708, a bag-in-bottle cap retainer 710, a media tube orifice 712, a seal housing(s) 713, and/or a mixed manifold outlet 714.

In FIG. 7B, an illustration of a stand-alone mix manifold is shown, according to one embodiment. A device 740 may include inlet quick connect dry break fitting 742, a water inlet check valve 744, a water outlet orifice 746, a media check valve retainer 748, a media tube orifice 750, a bag-in-bottle cap retainer 752, and/or a mixed manifold outlet 754.

The length of the tube orifice will dictate the flow rate of the active ingredient in the bag and therefore the ratio of the water to the active ingredient. For example, with Sanitizer A the tube orifice length is 13.5 inches based on the viscosity of Sanitizer A this will create a ratio of 514 to 1 (parts water to parts active) at a 0.50 oz. per second total mixed product flow rate and 341 to 1 (parts water to parts active) at a 0.9 oz. per second total mixed product flow rate. With Cleaner B the tube orifice length is 9.5 inches based on the viscosity of Cleaner B this will produce a ratio of 60 to 1 (parts water to parts active) at a 0.50 oz. per second total mixed product flow rate and a ratio of 32 to 1 (parts water to parts active) at a 0.90 oz. per second flow rate. If the ratio of water to active is to be reduced, the flow rate of the active is increased (stronger mixed product) by reducing the length of the tube orifice and if the ratio of water to active is to be increased, the flow rate of the active is reduced (weaker mixed product) by increasing the length of the tube orifice.

The interior pressure is the same on both canisters, but the flow rate is different on each, due to the length of the tube. This allows for different application rates but to be empty on the same day to minimize re-installation, i.e., both canisters are empty at the same time. In various example, the need to replace canisters is predictable, a certain day, X days hence. No continuing monitoring is necessary. There is no wasted labor and no human error.

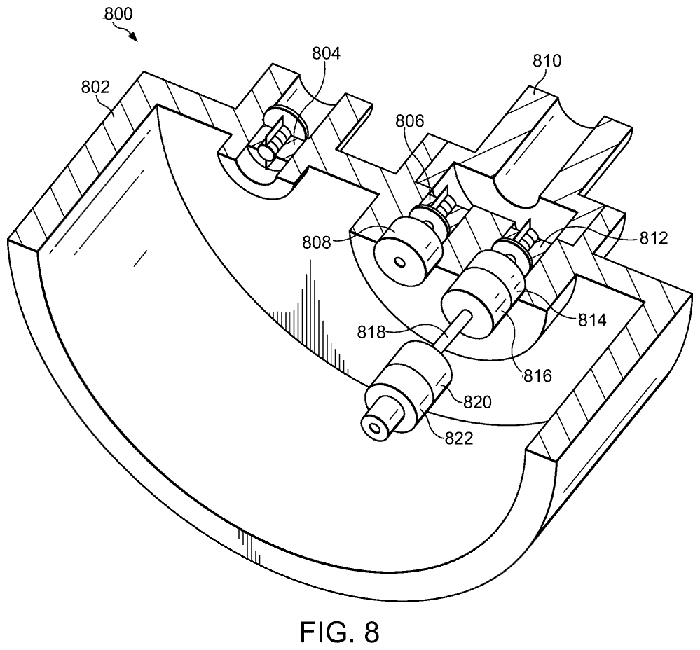

In FIG. 8, an illustration of a cap of a canister is shown, according to one embodiment. A cap may include a manifold cap 802, a water inlet check valve 804, a water outlet check valve 806, a water orifice check valve plug 808, a mixed manifold outlet 810, a media outlet check valve 812, a media check valve retainer 814, a seal housing 816, a media tube orifice 818, a seal housing 820 and/or a bag-in-bottle cap retainer 822.

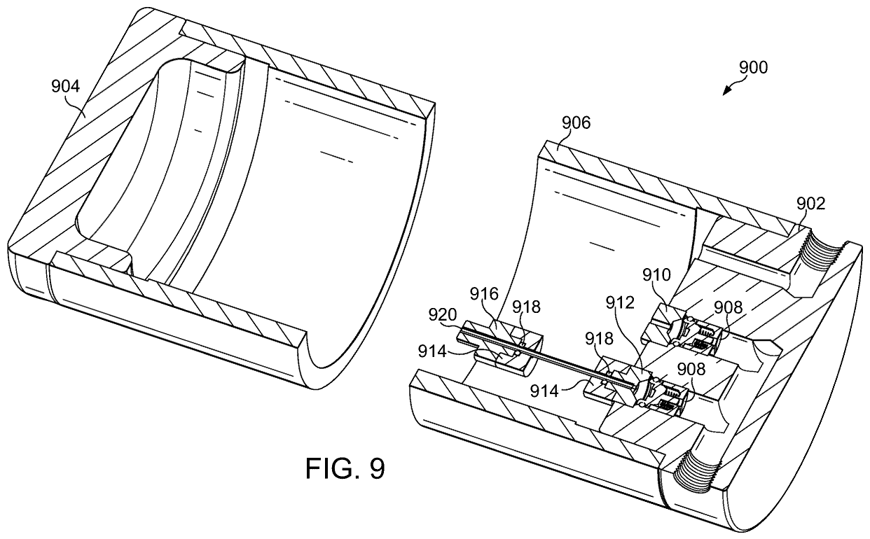

In FIG. 9, another illustration of a cap of a canister is shown, according to one embodiment. A cap 900 may include a 3″ SCH 40 CIP UHR glued Manifold plug 902, a 3″ SCH 40 CIP HUR glued end plug 904, a 3″ SCH 40 CIP UHR glued pipe bore 906, a check valve 908, a water orifice check valve plug 910, an UHR check valve retainer 912, an UHR seal housing 914, an UHR Bib Cap retainer 916, an O-ring 918, and/or an orifice tube 920.

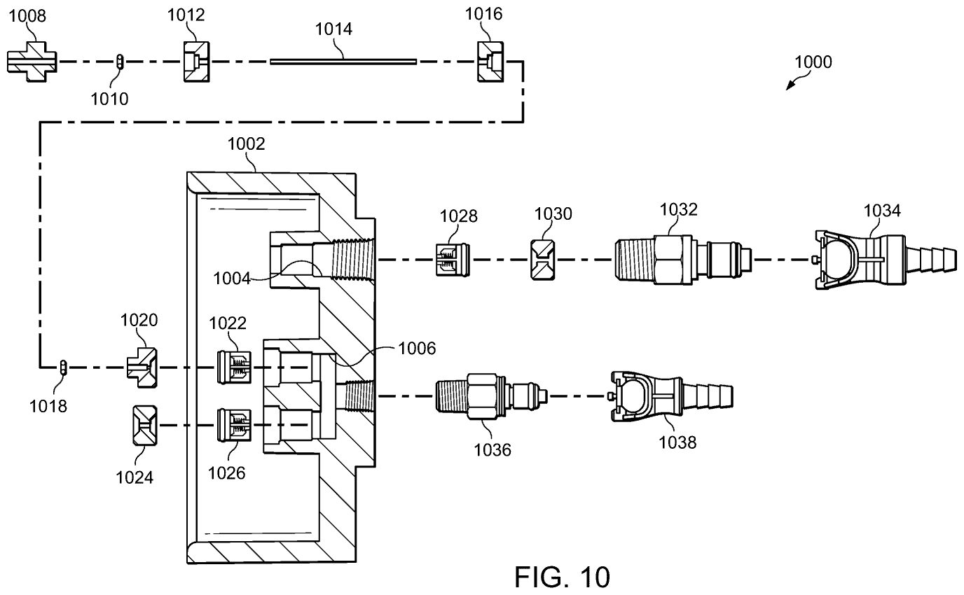

In FIG. 10, an illustration utilizing a CFValve (solenoid valve) is shown, according to one embodiment. A device 1000 may include a manifold cap 1002, a water inlet passage 1004, a mixed manifold outlet passage 1006, a bag-in-bottle cap retainer 1008, an O-ring 1010, a seal housing 1012, a media tube orifice 1014, a seal housing 1016, an O-ring 1018, a media check valve retainer 1020, a media check valve 1022, a water orifice check valve plug 1024, a water check valve 1026, a water inlet check valve 1028, a water inlet check valve retainer 1030, a water inlet quick connect dry break 1032 (e.g., male), a water inlet quick connect dry break 1034 (e.g., female), a mixed media outlet quick connect dry break 1036 (e.g., male), and/or a mixed media outlet quick connect dry break 1038 (e.g., female).

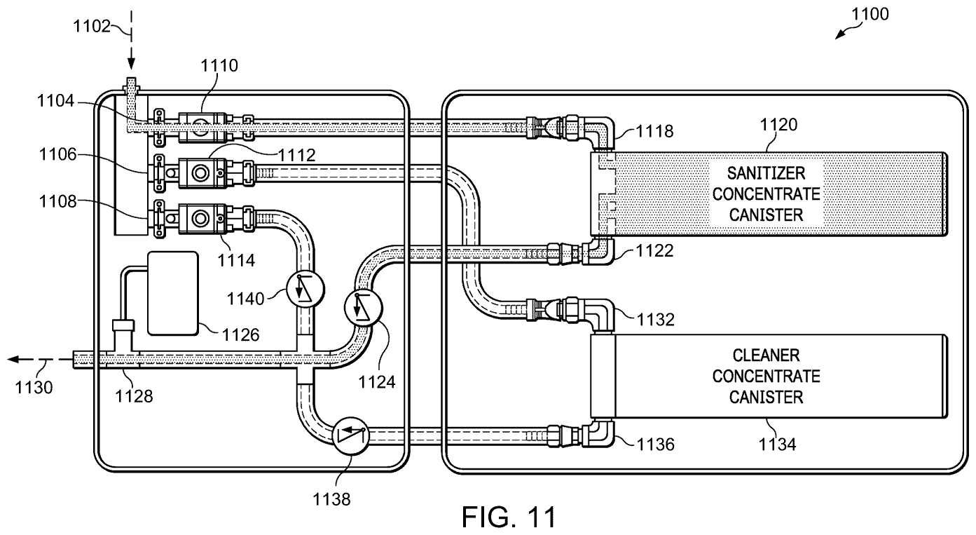

In FIG. 11, an illustration of a sanitation system is shown, according to one embodiment. A sanitation system 1100 may include a water supply inlet 1102, a sanitizer CFValve (solenoid valve) circuit 1104, a cleaner CFValve (solenoid valve) 1106 circuit, a water flush circuit 1108, a sanitizer CFValve (solenoid valve) 1110 (e.g., CFive), a cleaner CFValve (solenoid valve) 1112 (e.g., CFive), a water flush CFValve (solenoid valve) 1114 (e.g., CFive), a first water inlet dry break fitting 1118, a sanitizer concentrate canister 1120, a first diluted sanitizer outlet dry break fitting 1122, a first check valve 1124, a total dissolved solids (conductivity) controller 1126 with one or more sensors, a total dissolved solids (conductivity) TDS (conductivity) meter and sensors 1128, a diluted solution outlet 1130, a second water inlet dry break fitting 1132, a cleaner concentrate canister 1134, a diluted cleaner outlet dry break fitting 1136, a second check valve 1138, and/or an Nth check valve 1140. In various examples, the system can have only one canister or up to an Nth canister (e.g., 1-1,000).

The water source (i.e., city water) enters the system through and passes by a pressure regulator through a T the water is directed to the two CFiVes that control the UHR system, the CFiVe controls the pressure of the water to the targeted Pressure into the Canisters, the water enters the canister through the Manifold water inlet and pressurizes the bag of active ingredient which pushes active ingredient through the tube orifice into the mixing manifold where it mixes with water and flows back into the machine through the flow meter/total dissolved solids (conductivity) sensor and into the manifold via the incoming sanitation solenoid, one the cleaning or sanitation mixed solution is in the solenoid manifolds it then passes through each of the various solenoids downstream to sanitize/clean that particular circuit in the system.

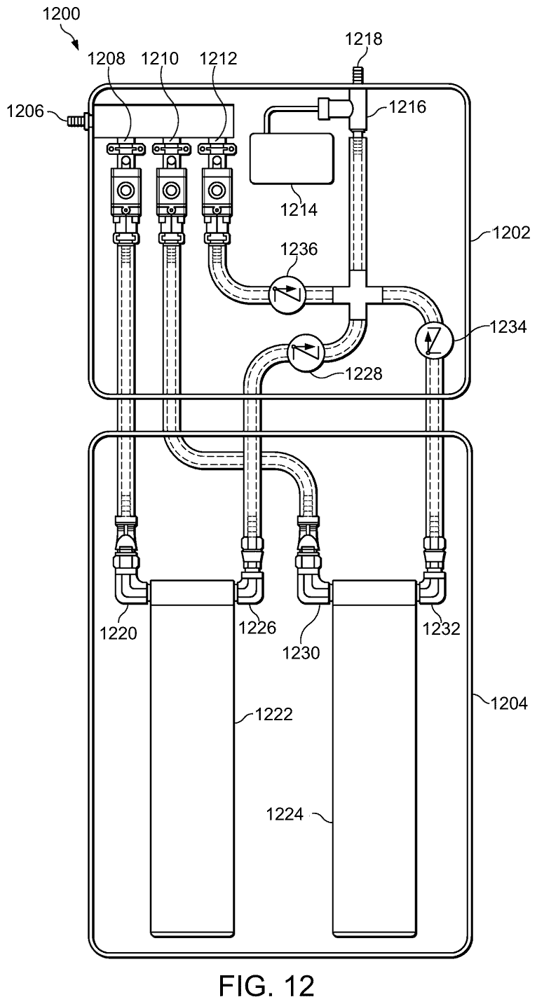

In FIG. 12, another illustration of a sanitation system is shown, according to one embodiment. A sanitization system 1200 may include a control box 1202 (and/or boxes) and a cartridge box 1204 (and/or cartridge boxes). The control box 1202 may include a water supply 1206, a sanitation canister controller 1208, a cleaner canister controller 1210, a flush controller 1212, a total dissolved solids (conductivity) controller 1214, one or more total dissolved solids (conductivity) sensors 1216, a first check valve 1228, a second check valve 1234, and an Nth check valve 1236. The cartridge box 1204 may include a sanitation canister water inlet area 1220, a sanitation canister 1222, a sanitation canister mixed product outlet area 1226, a cleaner canister water inlet area 1230, a cleaner canister 1224, and a cleaner canister mixed product outlet area 1232. It should be noted that any number of canisters may be utilized and that any product (e.g., vinegar, chemical A, natural product A, chemical B, etc.) may be in one or more canisters.

For ease of retrofit the cleaning canisters can be mounted outside the machine (behind, below, above), the same water that is utilized inside the machine can be routed out the machine and into a Tee into the CFiVes. When the CFive is actuated the water flows into the canister from the respective CFiVe, the mixed product exits the canister and flows to a T and into the back of the machine to the Solenoid Manifold inside the machine or into the chosen flow path (one or more) that will be cleaned/sanitized within the machine.

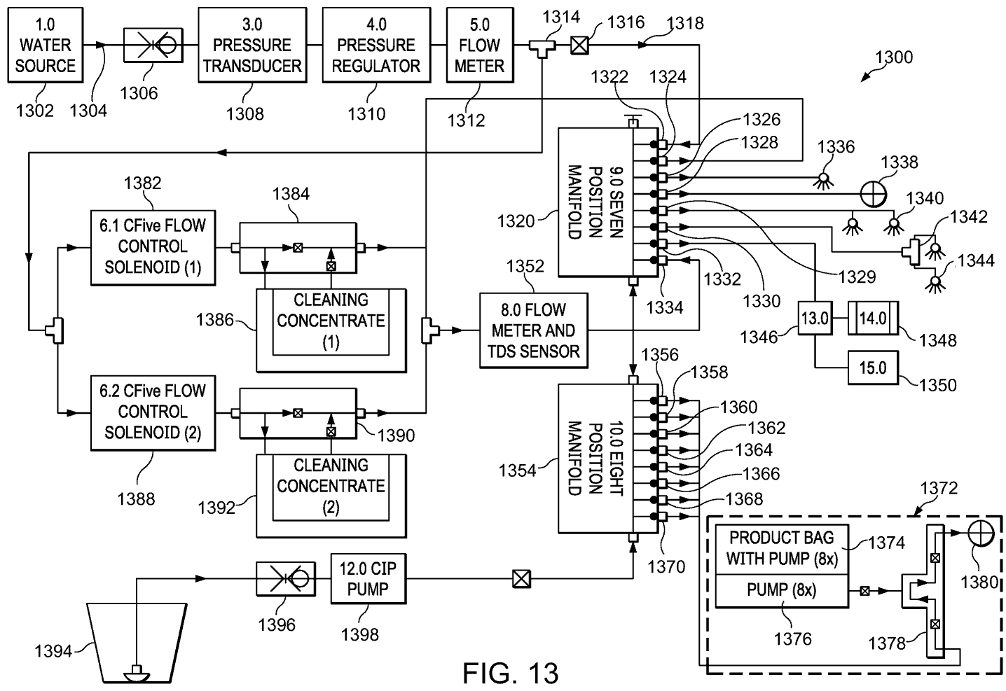

In FIG. 13, another illustration of a sanitation system is shown, according to one embodiment. A sanitation system 1300 may include a water source 1302, a first valve 1304, an inlet water fitting 1306, a pressure transducer 1308, a pressure regulator 1310, a flow meter 1312, a second valve 1316, a third valve 1318, a first manifold 1320 (with a first position 1322, a second position 1324, a third position 1326, a fourth position 1328, a fifth position 1329, a sixth position 1330, and a seventh position 1332), an incoming sanitation 1334, a spray head 1336, a LMS valve 1338, a spray head 1340, a T joint 1342, a spray head 1344, a first flow characteristic device 1346, a second flow characteristic device 1348, a third flow characteristic device 1350, a second manifold 1354 (with a first position 1356, a second position 1358, a third position 1360, a fourth position 1362, a fifth position 1364, a sixth position 1366, a seventh position 1368, and an eighth position 1370, a product dispensing line 1372, a product bag with pump 1374, a pump 1376, a T joint 1378, a LMS valve 1380, a CFive flow control solenoid 1382, a third manifold 1384, a flow meter and total dissolved solids (conductivity) sensors 1352, a cleaning concentrate 1386, a second CFive flow control solenoid 1388, a fourth manifold 1390, a second cleaning and/or sanitizer concentrate 1392, a sanitizer/cleaner line 1394, a sanitizer/cleaner fitting 1396, and/or a clean-in-place (CIP) pump 1398.

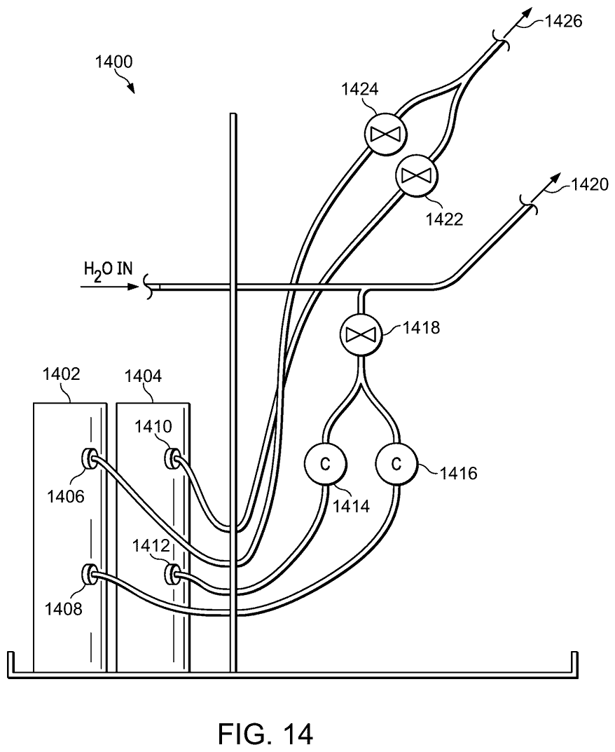

In FIG. 14, another illustration of a sanitation system is shown, according to one embodiment. A sanitation system 1400 may include a first canister 1402 (with concentrate), a second canister 1404 (with a different concentrate), a first canister outlet 1406, a first canister outlet 1408, a second canister outlet 1410, a second canister outlet 1412, a first flow control 1414, a second flow control 1416, a first check valve 1418, a second check valve 1422, an Nth check valve 1424, a drink line 1420, and a cleaning/sanitizer/flush line 1426. In one example, two out tubes and two in tubes can fit through the same holes.

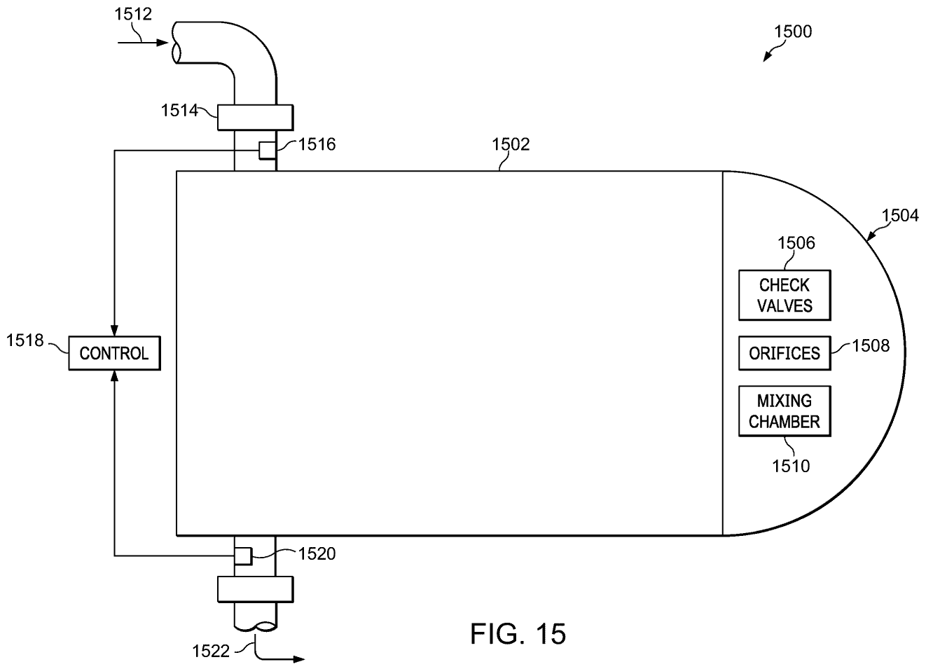

In FIG. 15, an illustration of a canister is shown, according to one embodiment. A canister 1500 may include a canister 1502, a cap 1504, one or more check valves 1506, one or more orifices 1508, a mixing chamber 1510, a water source 1512, a CFValve (solenoid valve) 1514, a first total dissolved solids (conductivity) sensor 1516, a controller 1518, a second total dissolved solids (conductivity) sensor 1520, and a mixture outlet area 1522. In one example, the one or more check valves 1506, the one or more orifices 1508 and the mixing chamber are inside of cap 1504.

The methods and/or methodologies described herein may be implemented by various means depending upon applications according to particular examples. For example, such methodologies may be implemented in hardware, firmware, software, or combinations thereof. In a hardware implementation, for example, a processing unit may be implemented within one or more application specific integrated circuits (“ASICs”), digital signal processors (“DSPs”), digital signal processing devices (“DSPDs”), programmable logic devices (“PLDs”), field programmable gate arrays (“FPGAs”), processors, controllers, micro-controllers, microprocessors, electronic devices, other devices designed to perform the functions described herein, or combinations thereof.

Some portions of the detailed description included herein are presented in terms of algorithms or symbolic representations of operations on binary digital signals stored within a memory of a specific apparatus or a special purpose computing device or platform. In the context of this particular specification, the term specific apparatus or the like includes a general-purpose computer once it is programmed to perform particular operations pursuant to instructions from program software. Algorithmic descriptions or symbolic representations are examples of techniques used by those of ordinary skill in the arts to convey the substance of their work to others skilled in the art. An algorithm is considered to be a self-consistent sequence of operations or similar signal processing leading to a desired result. In this context, operations or processing involve physical manipulation of physical quantities. Typically, although not necessarily, such quantities may take the form of electrical or magnetic signals capable of being stored, transferred, combined, compared, or otherwise manipulated. It has proven convenient at times, principally for reasons of common usage, to refer to such signals as bits, data, values, elements, symbols, characters, terms, numbers, numerals, or the like. It should be understood, however, that all of these or similar terms are to be associated with appropriate physical quantities and are merely convenient labels. Unless specifically stated otherwise, as apparent from the discussion herein, it is appreciated that throughout this specification discussions utilizing terms such as “processing,” “computing,” “calculating,” “determining” or the like refer to actions or processes of a specific apparatus, such as a special purpose computer or a similar special purpose electronic computing device. In the context of this specification, therefore, a special purpose computer or a similar special purpose electronic computing device is capable of manipulating or transforming signals, typically represented as physical electronic or magnetic quantities within memories, registers, or other information storage devices, transmission devices, or display devices of the special purpose computer or similar special purpose electronic computing device.

Reference throughout this specification to “one example,” “an example,” “embodiment,” and/or “another example” should be considered to mean that the particular features, structures, or characteristics may be combined in one or more examples. Any combination of any element in this disclosure with any other element in this disclosure is hereby disclosed. For example, an element on page 6 can be combined with any element in this document (e.g., an element from page 20).

While there has been illustrated and described what are presently considered to be example features, it will be understood by those skilled in the art that various other modifications may be made, and equivalents may be substituted, without departing from the disclosed subject matter. Additionally, many modifications may be made to adapt a particular situation to the teachings of the disclosed subject matter without departing from the central concept described herein. Therefore, it is intended that the disclosed subject matter is not limited to the particular examples disclosed.

The techniques described herein are exemplary and should not be construed as implying any particular limitation on the present disclosure. It should be understood that various alternatives, combinations and modifications could be devised by those skilled in the art. For example, steps associated with the processes described herein can be performed in any order, unless otherwise specified or dictated by the steps themselves. The present disclosure is intended to embrace all such alternatives, modifications and variances that fall within the scope of the appended claims.

The terms “comprises” or “comprising” are to be interpreted as specifying the presence of the stated features, integers, steps, or components, but not precluding the presence of one or more other features, integers, steps or components or groups thereof.

What is claimed is:

1. A cleaning system for a drink dispensing device comprising:
a cleaner canister coupled to a water source; and
a cleaner CFValve coupled to the water source which provides a first water flow to the cleaner canister, the cleaner canister configured to provide a cleaner solution to one or more parts of the drink dispensing device.

2. The cleaning system of claim 1, further comprising a sanitizer canister coupled to the water source and a sanitizer CFValve coupled to the water source which provides a second water flow to the sanitizer canister, the sanitizer canister configured to provide a sanitizer solution to one or more parts of the drink dispensing device.

3. The cleaning system of claim 2, further comprising a water flush device coupled to the water source and a water flush CFValve coupled to the water source which provides a third water flow to the one or more parts of the drink dispensing device.

4. The cleaning system of claim 2, further comprising an inlet dry breaking fitting and an outlet dry breaking fitting on the sanitizer canister.

5. The cleaning system of claim 1, further comprising an inlet dry breaking fitting and an outlet dry breaking fitting on the cleaner canister.

6. The cleaning system of claim 1, further comprising a total dissolved solids device which measures an inlet total dissolved solids and an outlet total dissolved solids.

7. The cleaning system of claim 1, further comprising:
a sanitizer canister coupled to the water source and a sanitizer CFValve coupled to the water source which provides a second water flow to the sanitizer canister, the sanitizer canister configured to provide a sanitizer solution to one or more parts of the drink dispensing device;
a water flush device coupled to the water source and a water flush CFValve coupled to the water source which provides a third water flow to the one or more parts of the drink dispensing device; and
a total dissolved solids device which measures an inlet total dissolved solids and an outlet total dissolved solids.

8. The cleaning system of claim 1, further comprising:
a sanitizer canister coupled to the water source and a sanitizer CFValve coupled to the water source which provides a second water flow to the sanitizer canister, the sanitizer canister configured to provide a sanitizer solution to one or more parts of the drink dispensing device;
a water flush device coupled to the water source and a water flush CFValve coupled to the water source which provides a third water flow to the one or more parts of the drink dispensing device;
a total dissolved solids device which measures an inlet total dissolved solids and an outlet total dissolved solids;
an inlet dry breaking fitting and an outlet dry breaking fitting on the sanitizer canister; an inlet dry breaking fitting and an outlet dry breaking fitting on the cleaner canister;
and
a controller that controls one or more ratios based on the inlet total dissolved solids and the outlet total dissolved solids.

9. The cleaning system of claim 8, wherein one or more of the cleaner CFValve, the sanitizer CFValve, and the water flush CFValve is configured to maintain a relative constant flow of fluid from a variable pressure fluid supply to a fluid outlet, the CF Valve including:
a) a valve housing having an inlet port and an outlet port adapted to be connected to the variable pressure fluid supply and the fluid outlet;
b) a diaphragm chamber interposed between the inlet port and the outlet port;
c) a cup contained within the diaphragm chamber;
d) a diaphragm closing the cup;
e) a piston assembly secured to a center of the diaphragm, the piston assembly having a cap and a base;
f) a stem projecting from the cap through a first passageway in a barrier wall to terminate in a valve head; and
g) a spring in the cup co-acting with the base of the piston assembly for urging the diaphragm into a closed position, and the spring being responsive to fluid pressure above a predetermined level to adjust a size of a control orifice.

10. The cleaning system of claim 8, wherein one or more of the cleaner CFValve, the sanitizer CFValve, and the water flush CFValve is configured to maintain a relative constant flow of fluid from a variable pressure fluid supply to a fluid outlet, the CF Valve including: a base having a wall segment terminating in an upper rim, and a projecting first flange; a cap having a projecting ledge and a projecting second flange, the wall segment of the base being located inside the cap with a space between the upper rim of the base and the projecting ledge of the cap; a barrier wall subdividing an interior of a housing into a head section and a base section; a modulating assembly subdividing the base section into a fluid chamber and a spring chamber; and
an inlet in the cap for connecting the head section to a fluid source; a port in the barrier wall connecting the head section to the fluid chamber, the port being aligned with a central first axis of the CF Valve; an outlet in the cap communicating with the fluid chamber, the outlet being aligned on a second axis transverse to the first axis; a stem projecting from the modulating assembly along the first axis through the port into the head section; a diaphragm supporting the modulating assembly within the housing for movement in opposite directions along the first axis, a spring in the spring chamber, the spring being arranged to urge the modulating assembly into a closed position at which the diaphragm is in sealing contact with the barrier wall, and the spring being responsive to fluid pressure above a predetermined level to adjust a size of a control orifice.
